SEQUENCE LISTING (17 sequences)

SEQ ID NO: 1 - DNA, 39 bp, artificial
  forward primer
ctgctctccaagcacagcatctatntncagnnnatcaac

SEQ ID NO: 2 - DNA, 57 bp, artificial
  reverse primer
gggggccaagcgnannnncnnnnnnnnnnnnnncacagttggnnngttgatnnnctg

SEQ ID NO: 3 - DNA, 48 bp, artificial
  forward primer
cgcttggccccctccccccaccacagccctcagatgatggaaaacttt

SEQ ID NO: 4 - DNA, 32 bp, artificial
  forward primer
atctgtgatcttctgctctccaagcacagcat

SEQ ID NO: 5 - DNA, 28 bp, artificial
  reverse primer
cttctccacaaagttttccatcatctga

SEQ ID NO: 6 - DNA, 20 bp, artificial
  forward primer
tgcaggccatagaggagacc

SEQ ID NO: 7 - DNA, 17 bp, artificial
  reverse primer
tacgagttgcatgataa

SEQ ID NO: 8 - protein, 16 aa, Homo sapiens
VQTINYPTVPRGEELL

SEQ ID NO: 9 - protein, 16 aa, artificial
  mutated ALAS sequence derived from Homo sapiens wild type
VQAINYPTVPRGEEQL

SEQ ID NO: 10 - protein, 16 aa, artificial
  mutated ALAS sequence derived from Homo sapiens wild type
VQAIHYPTVPRGEELL

SEQ ID NO: 11 - protein, 16 aa, artificial
  mutated ALAS sequence derived from Homo sapiens wild type
VQAINYPTVPKKQEQL

SEQ ID NO: 12 - protein, 16 aa, artificial
  mutated ALAS sequence derived from Homo sapiens wild type
VQAINIPTVPQNTENL

SEQ ID NO: 13 - protein, 16 aa, artificial
  mutated ALAS sequence derived from Homo sapiens wild type
IQPINCPTVRKGNELL

SEQ ID NO: 14 - protein, 16 aa, artificial
  mutated ALAS sequence derived from Homo sapiens wild type
VQGINHPTVPHNKEKL

SEQ ID NO: 15 - protein, 16 aa, artificial
  mutated ALAS sequence derived from Homo sapiens wild type
VQAINNPTVNIEKEKL

SEQ ID NO: 16 - protein, 16 aa, artificial
  mutated ALAS sequence derived from Homo sapiens wild type
LQAINRPTVEINQEKL

SEQ ID NO: 17 - protein, 16 aa, Homo sapiens
VQAINYPTVPRGEELL

(19) United States
(12) Patent Application Publication
(10) Pub. No.: US 2010/0248328 A1
(43) Pub. Date: Sep. 30, 2010

(54) HYPERACTIVE VARIANTS OF 5-AMINOLEVULINATE SYNTHASE AND METHODS OF USE

(75) Inventors: Gregory A. Hunter, Tampa, FL (US); Gloria C. Ferreira, Tampa, FL (US); Thomas Lendrihas, Temple Terrace, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, TAMPA, FL (US)

(21) Appl. No.: 12/691,359

(22) Filed: Jan. 21, 2010

Related U.S. Application Data
(63) Continuation-in-part of application No. 11/427,604, filed on Jun. 29, 2006, now abandoned.
(60) Provisional application No. 60/695,172, filed on Jun. 29, 2005.

(51) Int. Cl. C12N 9/10

(52) U.S. Cl. 435/193

(57) ABSTRACT

The rate of porphyrin biosynthesis in mammals is controlled by the activity of the pyridoxal 5′-phosphate-dependent enzyme 5-aminolevulinate synthase. Assuming the turnover in this enzyme is controlled by conformational dynamics at a highly conserved active site loop, a variant library was constructed by targeting imperfectly conserved non-catalytic loop residues and the effects on product and porphyrin production were examined. Functional loop variants of the enzyme were tested for porphyrin fluorescence, which varied widely and thus facilitated identification of clones encoding unusually active enzyme variants. Nine loop variants leading to high in vivo porphyrin production were purified and characterized kinetically. Steady-state catalytic efficiencies for the two substrates were increased by up to one hundred-fold. The data support the postulate that the active site loop controls the rate of product and porphyrin production in vivo and suggest the possibility of an as yet undiscovered means of allosteric regulation.

### CROSS REFERENCE TO RELATED APPLICATION

This application is a continuation-in-part of U.S. patent application Ser. No. 11/427,604, entitled “Therapeutic Applications of Aminolevulinate Synthase”, filed Jun. 29, 2006, which claims priority to U.S. Provisional Patent Application No. 60/695,172, entitled, “Therapeutic Applications of Aminolevulinate Synthase”, filed Jun. 29, 2005, the contents of which are herein incorporated by reference.

### STATEMENT OF GOVERNMENT SUPPORT

This invention was made with government support under Grant No. DK63191, awarded by the National Institutes of Health. The Government has certain rights in the invention.

### FIELD OF INVENTION

This invention relates to medical therapeutics, for use in diseases such as cancer, atherosclerosis and the prevention of restenosis following angioplasty. Specifically, the invention involves a procedure for the creation, identification, and use of a 5-aminolevulinate synthase variants conferring maximal photosensitivity upon a given cell population.

### BACKGROUND OF THE INVENTION

Tobacco smoking is the leading preventable cause of death in the United States. A well-defined correlation exists between exposure to tobacco smoke and several cancers, including lung, esophageal, bladder, larynx, and oral cancers. Exposure to tobacco smoke also promotes coronary artery atherosclerosis, and is the main cause of abdominal aortic aneurysms. Photodynamic therapy (PDT) is a treatment that is based upon the differential uptake by cancerous or other target cells of photosensitizing agents, followed by irradiation of the cells to cause a photochemical reaction. PDT involves the administration of a photosensitizing agent to target cells, which may accumulate in the target cells, and subsequent irradiation with light of the target cells or tissue of the subject. These target cells or tissues are characterized by rapid proliferation or growth relative to the surrounding cells or tissues in the target environment.

PDT protocols for the treatment of disease typically involve the administration of a photosensitizer from about 15 minutes to about 2 days prior to the application of light. This allows the photosensitizer time to accumulate in the target disease tissue, while being cleared from normal tissue. Concerns in the use of the treatment include its safety and effectiveness. For example, PDT can have associated side-effects, such as development of inflammation with edema and pain, and even necrosis with scarring, at the target site. With systemically-delivered photosensitizers formulated in either aqueous or organic solvents, or in liposomal formulations, the side effects can include headaches, nausea, and fever, as well as skin photosensitivity. Moreover, the greater the dosage of photosensitizers used, the greater the risk of these, and potentially other, side effects. However, if too little photosensitizer is used in the treatment, then there is a greater risk of having only a partial response to treatment or recurrence of disease. While PDT shows great promise in the treatment of these tobacco related diseases, complete remission of disease using photodynamic therapy is often difficult to achieve.

Aminolevulinic acid (ALA) has been identified as a useful photosensitizing agent precursor. ALA is the universal building block of tetrapyrrole biosynthesis (Jordan, P. M. (1991) Biosynthesis of Tetrapyrroles, Elsevier, Amsterdam). ALAS is classified as a fold-type I PLP-dependent enzyme, and like the evolutionarily related L-amino acid transaminases (Eliot & Kirsch, Pyridoxal phosphate enzymes: mechanistic, structural, and evolutionary considerations. Annu Rev Biochem. 2004; 73:383-415), functions as a homodimer wherein a PLP cofactor is bound at each of the two active sites, which are recessed in clefts at the subunit interface (Astner, et al. Crystal structure of 5-aminolevulinate synthase, the first enzyme of heme biosynthesis, and its link to XLSA in humans. EMBO J. 2005 Sep. 21; 24(18):3166-77. Epub 2005 Aug. 25; Tan & Ferreira, Active site of 5-aminolevulinate synthase resides at the subunit interface. Evidence from in vivo heterodimer formation. Biochemistry. 1996 Jul. 9; 35(27):8934-41). Most of the photosensitizers reported so far fall into the two broad classes of porphyrins and porphyrin precursors based on ALA. The systemic or topical administration of ALA or ALA esters results in transient accumulation of protoporphyrin IX, which is a good photosensitizer with an excellent pharmacokinetic profile relative to most other photosensitizers. (Webber, J., et al. (1997) J Surg Res 68:31-7; Dalton, J. T., et al. (2002) J Pharmacol Exp Ther 301:507-12; Fukuda, H., et al. (2005) Int J Biochem Cell Biol 37:272-6) Protoporphyrin IX accumulates because ALAS is the rate-limiting enzyme of the porphyrin biosynthetic pathway. Protoporphyrin IX is not efficiently converted into heme (which is an ineffective photosensitizer) under these conditions presumably because porphyrin synthesis has been uncoupled from iron delivery by circumventing the natural regulation of ALAS activity. This results in the rate of porphyrin synthesis outpacing the rate of iron deliver to the enzyme ferrochelatase, which catalyzes conversion of protoporphyrin IX and iron into heme, and consequently protoporphyrin IX accumulates.

In non-plant eukaryotes and the α-subclass of purple bacteria the production of ALA is catalyzed by the pyridoxal 5′-phosphate (PLP)-dependent enzyme 5-aminolevulinate synthase (ALAS) (EC 2.3.1.37), in a reaction involving the Claisen-like condensation of succinyl-Coenzyme-A and glycine to yield CoA, carbon dioxide and ALA (Lendrihas, et al. Arg-85 and Thr-430 in murine 5-aminolevulinate synthase coordinate acyl-CoA-binding and contribute to substrate specificity. Protein Sci. 2009 September; 18(9):1847-59). ALAS catalyzes the first committed step of tetrapyrrole biosynthesis in these organisms, which is also the rate-determining step of the pathway. Consequently, overexpression of ALAS in prokaryotic and eukaryotic cells results in accumulation of the photosensitizing heme precursor protoporphyrin IX (Whatley, et al. C-terminal deletions in the ALAS2 gene lead to gain of function and cause X-linked dominant protoporphyria without anemia or iron overload. Am J Hum Genet. 2008 September; 83(3):408-14. Epub 2008 Sep. 4). This property could potentially lead to novel applications of ALAS or ALAS variants in photodynamic therapy of tumors and other dysplasias (Gagnebin, et al. A photosensitising adenovirus for photodynamic therapy. Gene Ther. 1999 October; 6(10):1742-50).

In homo sapiens, ALAS-1 (sometimes referred to as the “housekeeping” ALAS) is expressed in all tissues, with the highest levels occurring in the liver, where it appears to function in the production of heme for cytochromes. ALAS-2, on the other hand, is expressed almost exclusively in developing erythrocytes, where it supports heme synthesis for hemoglobin production. The erythroid specific ALAS contains an iron-responsive-element in the 5′-untranslated region that inversely coordinates iron availability with ALAS-2 mRNA translation, but this regulatory element is not present in the housekeeping ALAS mRNA (Cox, et al. Human erythroid 5-aminolevulinate synthase: promoter analysis and identification of an iron-responsive element in the mRNA. EMBO J. 1991 July; 10(7):1891-902; Dandekar, et al. Identification of a novel iron-responsive element in murine and human erythroid delta-aminolevulinic acid synthase mRNA. EMBO J. 1991 July; 10(7):1903-9). Mitochondrial import of mammalian ALAS isoforms is essential because the substrate succinyl-CoA is produced in the mitochondrial matrix, and both isoforms contain multiple heme regulatory motifs in the protein presequence that support heme feedback inhibition of mitochondrial import (Lathrop & Timko. Regulation by heme of mitochondrial protein transport through a conserved amino acid motif. Science. 1993 Jan. 22; 259(5094):522-5). It is possible to eliminate this feedback regulation by mutating the cysteine residues present in the heme regulatory motifs to alanine or serine, with the result that translocation into the mitochondrial matrix occurs even at elevated heme concentrations (Gagnebin, J., et al. A photosensitising adenovirus for photodynamic therapy. Gene Ther. 1999 October; 6(10):1742-50; Lathrop & Timko Regulation by heme of mitochondrial protein transport through a conserved amino acid motif. Science. 1993 Jan. 22; 259(5094):522-5). Upon mitochondrial import the protein presequence is removed to form the mature ALAS enzyme, which appears to associate with the matrix side of the inner mitochondrial membrane. The erythroid specific ALAS may form a functional complex with succinyl-CoA synthetase, although such complexation is not absolutely required for catalytic activity, and the housekeeping ALAS does not complex succinyl-CoA synthetase. (Furuyama, K., et al. (2000) J Clin Invest 105:757-64).

X-ray crystal structures of ALAS from Rhodobacter capsulatus and the closely related enzyme 8-amino-7-oxononanoate synthase from E. coli reveal an induced fit type mechanism wherein binding of substrates and product, respectively, trigger closure of an extended loop over the active site (Astner, et al. Crystal structure of 5-aminolevulinate synthase, the first enzyme of heme biosynthesis, and its link to XLSA in humans. EMBO J. 2005 Sep. 21; 24(18):3166-77. Epub 2005 Aug. 25; Webster, et al. Mechanism of 8-amino-7-oxononanoate synthase: spectroscopic, kinetic, and crystallographic studies. Biochemistry. 2000 Jan. 25; 39(3):516-28), seen in FIGS. 11(A) and 11(B). The inferred conformational dynamics of this loop are of interest because kinetic and crystallographic studies support the hypothesis that the rate of ALA, and hence protoporphyrin IX, production is controlled by opening of the active site loop coincident with product release (Astner, et al. Crystal structure of 5-aminolevulinate synthase, the first enzyme of heme biosynthesis, and its link to XLSA in humans. EMBO J. 2005 Sep. 21; 24(18):3166-77; Hunter, & Ferreira. Pre-steady-state reaction of 5-aminolevulinate synthase. Evidence for a rate-determining product release J Biol. Chem. 1999 Apr. 30; 274(18):12222-8; Hunter, et al. Transient kinetic studies support refinements to the chemical and kinetic mechanisms of aminolevulinate synthase. J Biol. Chem. 2007 Aug. 10; 282(32):23025-35. Epub 2007 May 7). This mechanism, if correct, is notable in that the catalytic rate, which is 0.66/s at 37° C., is slow in comparison to similar conformational motions measured in other enzymes (Benkovic & Hammes-Schiffer. A perspective on enzyme catalysis. Science. 2003 Aug. 29; 301(5637):1196-202), suggesting that the primary selective pressures for ALAS evolution are oriented away from speed.

Although not intending to be bound by any particularly theory, it has been proposed that following administration of ALA, as a result of their more rapid proliferation, the target cells or tissue contain relatively greater concentrations of light sensitive porphyrins and thus are more sensitive to light. The photochemical reaction is believed to generate chemically disruptive species, such as singlet oxygen. These disruptive species in turn injure the cells through reaction with cell parts, such as cellular and nuclear membranes. Complicating matters, in vitro studies demonstrate that production of protoporphyrin IX from ALA is not only concentration dependent, but also cell line dependent. (Tsai, et al. Effect of 5-aminolevulinic acid-mediated photodynamic therapy on MCF-7 and MCF-7/ADR cells. Lasers Surg Med. 2004; 34(1):62-72; Chakrabarti, et al. Delta-aminolevulinic acid-mediated photosensitization of prostate cell lines: implication for photodynamic therapy of prostate cancer. Prostate. 1998 Sep. 1; 36(4):211-8; Bartosova, et al. Accumulation of protoporphyrin-IX (PpIX) in leukemic cell lines following induction by 5-aminolevulinic acid (ALA). Comp Biochem Physiol C Toxicol Pharmacol. 2000 July; 126(3):245-52. 126:245-52; Tsai, et al. Comparative study on the ALA photodynamic effects of human glioma and meningioma cells. Lasers Surg Med. 1999; 24(4):296-305). The mechanisms behind the cell type specific response to a given concentration of ALA could include varying degrees of cellular ALA uptake, as well as varying levels of the enzymes subsequent to ALAS in the biosynthetic pathway to protoporphyrin IX. Different cell types also have different intrinsic photosensitivities, possibly due to differences in susceptibility to oxidative damage. These data suggest that although a targeting strategy might circumvent the issue of differential cellular ALA uptake, the maximal levels of protoporphyrin IX achievable may differ depending upon the cells targeted and thereby limit the applicability of ALA-PDT to only those cell types capable of producing sufficient protoporphyrin IX to induce cell death upon illumination. Regardless, PDT has been used successfully for treating several types of cancer cells.

Due to the limitations in PDT photosensitizers, like edema, pain, necrosis and scarring, there exists a needed to develop compounds and methods to allow the more selective accumulation of photosensitizer in the target cells relative to surrounding tissue. Such a selective accumulation would enable the effective treatment of the diseased target area while limiting collateral effects.

### SUMMARY OF THE INVENTION

The present invention offers one possible way to achieve high-level selective photosensitizer accumulation. By identifying and delivering to target cells the enzymatic capacity to hyper-stimulate protoporphyrin IX production, the selective photosensitizer accumulation could conceivably be elevated to levels not previously possible, with resulting increases in efficacy of treatment and expansion of treatable populations.

One way to enhance the efficacy of photodynamic therapy regimes would be to provide a means for the selective accumulation of photosensitizing agent within the target tissues to levels not currently possible. The painful side-effect of photosensitivity necessitates that these increased concentrations be delivered in as localized a manner as feasible.

This work builds on the assumption that if loop dynamics were a primary determinant of the slow catalytic rate, then it should be straightforward to generate hyperactive ALAS variants by targeting non-conserved residues in the active site loop. Besides shedding light on the mechanism of ALA production, hyperactive ALASs might also find philanthropic utility in generation of ALA for a variety of clinical and agricultural applications (Gagnebin, et al. A photosensitising adenovirus for photodynamic therapy. Gene Ther. 1999 October; 6(10):1742-50; Sasaki, et al. Biosynthesis, biotechnological production and applications of 5-aminolevulinic acid, Appl Microbiol Biotechnol. 2002 January; 58(1):23-9; Fukuda, et al. Aminolevulinic acid: from its unique biological function to its star role in photodynamic therapy. Int J Biochem Cell Biol. 2005 February; 37(2):272-6; Kelty, et al. The use of 5-aminolaevulinic acid as a photosensitiser in photodynamic therapy and photodiagnosis. Photochem Photobiol Sci. 2002 March; 1(3):158-68). To test the hypothesis, synthetic mixed base oligonucleotides were employed to generate a library of ALAS loop variants, coupled with a simple screening strategy to isolate potentially hyperactive clones.

### BRIEF DESCRIPTION OF THE DRAWINGS

For a fuller understanding of the invention, reference should be made to the following detailed description, taken in connection with the accompanying drawings, in which:

FIGS. 1(A1) through (B3) are images showing differential fluorescence of ALAS variant isolates streaked on expression agar. DAPI visualized cells containing: Wild-type ALAS (A1), and hyperactive variant SS2 (B1). Cells visualized for red fluorescence containing: Wild-type ALAS (A2), and hyperactive variant SS2 (B2). Overlay of DAPI and red fluorescence visualized cells: Wild-type ALAS (A3), and hyperactive variant SS2 (B3).

FIG. 2 is a table showing the alignment of sixty-five different species specific ALAS active site loop sequences with the hyperactive variants isolated in this study. The naturally occurring amino acid sequences were obtained from public databases (NCBI) using a BLAST search and aligned using CLUSTAL W (4). The 10 positions within the 18-amino acid sequence targeted for mutagenesis are high-lighted in cyan, and changes observed in hyperactive variants are high-lighted in magenta. The amino acid numbering in red refers to that of murine erythroid ALAS (mALAS-2). Represented proteins are: ALAS_DELLEU: Delphinapterus leucas ALAS (20138447); ALAS_DLLLEU: Delphinapterus leucas cook inlet subspecies 2 (5281116); ALAS_HOMSAP, Homo sapiens ALAS erythroid (4557299); ALAS_RATNOR, Rattus norvegicus erythroid ALAS (51980582); ALAS_RATRAT, Rattus rattus erythroid ALAS (6978485); ALAS2_MUSMUS, Mus musculus erythroid ALAS (33859502); ALAS_DANRER, Danio rerio ALAS (18858263); ALAS_DANROS, Danio roseus ALAS (20138448); ALAS_OPSTAU, Opsanus tau ALAS (1170202); ALAS_ORYLAT, Oryzias latipes ALAS (49022596); ALAS_HOMSPN, Homo sapiens erythroid ALAS (4502025); ALAS_DELDEL, Delphinus delphis erythroid ALAS (20138445); ALAS_MOUDOM, Mus musculus domesticus erythroid ALAS (23956102); ALAS_GALVAR, Gallus varius erythroid ALAS (122821); ALAS_XENLAE, Xenopus laevis erythroid ALAS (44968228); ALAS_OPSBET, Opsanus beta ALAS (1170206); ALAS_OPSPAR, Opsanus pardus ALAS (532630); ALAS_DANDAN, Danio danglia ALAS (32451642); ALAS2_MYXGLU, Myxine glutinosa erythroid ALAS (4433550); ALAS 1_BRALAN, Branchiostoma lanceolatum ALAS1 (28630217); ALAS 1_STRDRO, Strongylocentrotus droebachiensis ALAS1 (4433548); ALAS 1_DROMEL, Drosophila melanogaster ALAS1 (2330591); LAS 1_LIMPOL, Limulus polyphemus ALAS1 (4433540); ALAS 1_SEPOFF, Sepia officinalis ALAS1 (4433546); ALAS2_GLYDIB, Glycera dibranchiate ALAS 2 (4433544); ALAS2_GALGAL, Gallus gallus gallus ALAS 2 (1170201); ALAS_SINMEL, Sinorhizobium meliloti 1021 ALAS (15966742); ALAS_SMRMEL, Sinorhizobium meliloti ALAS (18266808); ALAS_AGRTUM, Agrobacterium tumefaciens ALAS (889869); ALAS_AGRRAD, Agrobacterium radiobacter ALAS (95069); ALAS_AGRTUM, Agrobacterium tumefaciens ALAS (122818); ALAS_RHOPAL, Rhodopseudomonas palustris ALAS (4001678); ALAS_RHOSPO, Rhodobacter sporagenes ALAS (541302); ALAS_EUGGRA, Euglena gracilis ALAS (12620813); ALAS_BRAELK, Bradyrhizobium elkanii ALAS (66534); ALAS_BRAJAP, Bradyrhizobium japonicum ALAS (30179569); ALAS_BRUMEL, Brucella melitensis ALAS (25286547); ALAS_ZYMMOB, Zymomonas mobilis ALAS (4511998); ALAS_RHOGLU, Rhodobacter gluconicum ALAS (97435); ALAS_RHOCAP, Rhodobacter capsulatus ALAS (122828); ALAS_PARDEN, Paracoccus denitrificans ALAS (1170207); ALAS_PARZEA, Paracoccus zeaxanthinifaciens ALAS (537435); ALAS_RHOSPH, Rhodobacter sphaeroides ALAS (541301); ALAS_EMENID, Emericella nidulans ALAS (418756); ALAS_EMCNID, Emericella nidulans ALAS (585244); ALAS_ASPNID Aspergillus nidulans ALAS (407-45239); ALAS_ASPORY, Aspergillus oryzae ALAS (5051989); ALAS_NEUCRA, Neurospora crassa ALAS (52782908); ALAS_GIBFUJ, Gibberella fujikuroi ALAS (15721883); ALAS_YARLIP, Yarrowia lipolytica ALAS (52782857); ALAS_CANBER, Candida berate ALAS (7493758); ALAS_CANALB, Candida albicans ALAS (10720014); ALAS_DEBHAN, Debaryomyces hansenii ALAS (52782855); ALAS_SACCER, Saccharomyces cerevisiae ALAS (6320438); ALAS_SACCAS, Saccharomyces castellii ALAS (122831); ALAS_CANGLA, Candida glabrata ALAS (52782865); ALAS_KLULAC, Kluyveromyces lactis ALAS (52788271); ALAS_EREGOS, Eremothecium gossypii ALAS (52782894); ALAS_ASPBIS, Agaricus bisporus ALAS (1679599); ALAS_AGADIV, Agaricus divoniensus ALAS (2492846); ALAS_SCHPOM, Schizosaccharomyces pombe ALAS (7492336); ALAS_SCHMIK, Schizosaccharomyces mikatae ALAS (52782853); ALAS_RICCON, Rickettsia conorii ALAS (7433712); ALAS_RICTYP, Rickettsia typhi ALAS (51474008); ALAS_RICPRO, Rickettsia prowazekii ALAS (6225494); ALAS_RICRIC, Rickettsia rickettsia ALAS (2528635); ALAS_CHRVIO, Chromobacterium violaceum ALAS (34102112).

FIG. 3 is illustration showing the generation and screening of the library. A library of over 100,000,000 possible ALAS variants was constructed with PCR using a series of degenerate mixed base oligonucleotides. The PCR product was ligated into an expression vector. (Ferreira & Dailey. Expression of mammalian 5-aminolevulinate synthase in Escherichia coli. Overproduction, purification, and characterization. J Biol. Chem. 1993 Jan. 5; 268(1):584-9). The resulting plasmids were transformed into Escherichia coli strain HU227 and plated on LB+ampicillin agar with and without ALA. The colonies that grew in the absence of ALA were identified as functional variants. Functional variants were screened for porphyrin overproduction by fluorescence microscopy.

FIG. 4 is a graph showing the single turnover reactions of isolated hyperactive ALAS variants. The wild-type enzyme single-turnovers occur in three steps; decarboxylation of a PLP-bound α-amino-β-ketoadipate intermediate to form a PLP-ALA quinonoid intermediate; protonation of the quinonoid intermediate to form the bound product; and opening of the active site loop coincident with product released.

FIGS. 5(A) through (D) are graphs showing the single turnover reactions of isolated hyperactive ALAS variants. The pre-steady state kinetic parameters were determined under single turnover conditions (60 μM enzyme-glycine complex, 10 μM succinyl-CoA) at 20° C. by monitoring absorbance changes at 510 nm. The variant catalyzed reactions A4, D8, G7, A8, (panels A-D, respectively) resemble that of the wild-type enzyme in that quinonoid lifetime is observed as a single step of quinonoid intermediate formation and 2-step process of decay (Hunter, & Ferreira. Pre-steady-state reaction of 5-aminolevulinate synthase. Evidence for a rate-determining product release J Biol. Chem. 1999 Apr. 30; 274(18):12222-8).

FIGS. 6(A) through (D) are graphs showing the single turnover reactions of isolated hyperactive ALAS variants. The pre-steady state kinetic parameters were determined under single turnover conditions (60 μM enzyme-glycine complex, 10 μM succinyl-CoA) at 20° C. by monitoring absorbance changes at 510 nm. The variant catalyzed reactions F3 and F10 (panels A and B, respectively) resemble that of the wild-type enzyme in that quinonoid lifetime is observed as a single step of quinonoid intermediate formation and 2-step process of decay (Hunter, & Ferreira. Pre-steady-state reaction of 5-aminolevulinate synthase. Evidence for a rate-determining product release J Biol Chem. 1999 Apr. 30; 274(18):12222-8). The reaction kinetics of the F1 and H1 variants (panels C and D respectively) are markedly altered in that the second of two steps defining quinonoid intermediate decay is not observed.

FIG. 7 is a graph showing the SS2 variant catalyzed reaction. Equilibrium and pre-steady state kinetic parameters for the SS2 variant were determined at 20° C. by monitoring changes in absorbance at 510 nm associated with quinonoid intermediate lifetime, under single-turnover conditions.

FIGS. 8(A) through (B) are graphs showing the SS2 variant catalyzed reaction. Equilibrium and pre-steady state kinetic parameters for the SS2 variant were determined at 20° C. by: (A) monitoring the change in absorbance at 420 nm (internal aldimine), and; (B) monitoring the change in fluorescence emission at 428 nm upon excitation at 330 nm following addition of ALA.

FIG. 9 is a graph showing the thermal dependence of the SS2 variant-catalyzed reaction. _{kcat} values were calculated by performing steady-state kinetics at different temperatures. (A) an Arrhenius plot depicting the difference between the wild-type () and the SS2 variant-catalyzed (◯) reactions at 288, 293, 298, 303 and 308K. Error bars are plotted over and partially obscured by the data points.

FIGS. 10(A) and (B) are chemical reactions showing the simulated kinetic mechanism of the SS2 variant-catalyzed reaction. Transition from an internal aldimine with lysine 313 to an external aldimine with glycine is the first step. Succinyl-CoA binds second which is followed by quinonoid formation, protonation of the quinonoid to yield an aldimine bound molecule of ALA. Finally, the release of ALA completes the reaction. E; SS2: G; glycine: EG; enzyme-glycine complex: SCoA; Succinyl-CoA: EGSCoA; ALAS-glycine-Succinyl-CoA complex: EQ; enzyme bound to protonated quinonoid: EALA; ALAS-ALA internal aldimine: EALA1; ALAS-ALA internal aldimine with active site loop closed: EALA2; ALAS-ALA internal aldimine with active site loop open.

FIGS. 11(A) and (B) are images of the position of the active site loop in the R. capsulatus ALAS crystal structure. In (A), the ribbon representations of the 3D structures of one monomer of holoenzymic (dark gray ribbon) and succinyl-CoA-bound (light gray) ALAS from R. capsulatus are superimposed. Note the distinct positioning of the active site loop in the two structures (depicted in dark gray in the ALAS holoenzyme structure and in dark gray in the succinyl-CoA-bound structure). In (B) the active site loop in the closed conformation of ALAS (i.e., succinyl-CoA-bound structure) is perched above the catalytic cleft of the enzyme. Succinyl-CoA and the co-factor PLP are shown as sticks. The image was constructed using Pymol and PDB entries 2BWN and 2BWO.

### DETAILED DESCRIPTION OF THE PREFERRED EMBODIMENT

The present invention provides an enhanced system of photodynamic therapy employing 5-aminolevulinate synthase (ALAS) variants. Photodynamic therapy (PDT) with 5-aminolevulinic acid is a promising approach to dysplastic disorders such as lung, esophageal, and bladder cancers, as well as atherosclerosis. PDT involves the local or systemic delivery of 5-aminolevulinate, which is then converted by natural cellular metabolism to the photosensitizing agent protoporphyrin IX. Upon light irradiation in the presence of dissolved oxygen, protoporphyrin IX catalyzes generation of reactive oxygen species and thereby stimulates apoptosis or necrosis in the light exposed cell. Although some cancers and plaque selectively accumulate protoporphyrin IX, the efficacy of photodynamic therapies in vitro can be dramatically enhanced by utilizing receptor mediated targeting to increase the accumulation of photosensitizer within the diseased cells.

In this regard recent effort has been directed towards developing the area of targeted PDT, which utilizes receptor-ligand interactions to drive selective accumulation of photosensitizer within discrete cell populations. (Sharman, et al., Targeted photodynamic therapy via receptor mediated delivery systems. Adv Drug Deliv Rev. 2004 Jan. 13; 56(1):53-76; Demidova, et al. Macrophage-targeted photodynamic therapy. Int J Immunopathol Pharmacol. 2004 May-August; 17(2): 117-26; Vrouenraets, et al. Targeting of aluminum (III) phthalocyanine tetrasulfonate by use of internalizing monoclonal antibodies: improved efficacy in photodynamic therapy. Cancer Res. 2001 Mar. 1; 61(5):1970-5) It is expected that this approach will enhance efficacy by realizing higher target tissue photosensitizer concentrations, while potentially resulting in enhanced safety profiles due to the lower quantities of drug required to elicit a therapeutic response.

Enhancing the specific activity of ALAS is considered essential to realize the optimal effectiveness of this approach because of the unusually low specific activity of this enzyme. The catalytic efficiency for the substrate glycine in particular, is only 3.4 mM⁻¹ min⁻¹ at 37 C and pH 7.2. (Tan, et al. Role of arginine 439 in substrate binding of 5-aminolevulinate synthase. Biochemistry. 1998 Feb. 10; 37(6):1478-84) for murine erythroid ALAS (k_{cat}/K_{M} for the other substrate, succinyl-CoA, is 20 μM⁻¹ min⁻¹ under similar conditions), meaning that even at glycine concentrations as high as 2 mM it still takes over a minute for one active site to produce the eight ALA molecules required to synthesize just one molecule of the photosensitizing agent protoporphyrin IX.

The in-vitro adenovirus delivery of an erythroid ALAS cDNA construct was previously modified by replacing the conserved cysteines in three HRMs to serines, which eliminated regulation by iron and heme, and infection with this construct resulted in sufficient ALA production to partially stimulate PDT-mediated apoptosis of H1299 lung carcinoma cells. However, addition of desferrioxamine was necessary for an optimal response, and potentiated photosensitivity nearly two-fold. (Gagnebin, et al. A photosensitising adenovirus for photodynamic therapy. Gene Ther. 1999 October; 6(10):1742-50) Desferrioxamine is an iron chelator that inhibits conversion of protoporphyrin IX into heme (Choudry, et al. The effect of an iron chelating agent on protoporphyrin IX levels and phototoxicity in topical 5-aminolaevulinic acid photodynamic therapy. Br J. Dermatol. 2003 July; 149(1):124-30), and the requirement for desferrioxamine in this study suggests that this approach did not result in a sufficient rate of ALA production to overwhelm the relatively slow rate of endogenous conversion of protoporphyrin IX to heme, and that better results might be obtained by increasing the ALAS activity delivered to the cells. The present invention provides a novel approach to PDT by constructing genetically diverse libraries of ALAS variants, and isolating individual variants from these libraries based on capacity to stimulate protoporphyrin IX production in the cells to be targeted for PDT. These variants result in the selective accumulation of protoporphyrin IX in and immediately around a target cell population to a degree not previously possible, and will lead to improvements in the effectiveness of current PDT practice in treating a variety of diseases including tobacco related diseases.

5-Aminolevulinic acid is also known as 5-aminolaevulinic acid, 6-aminolevulinic acid, δ-aminolaevulinic acid and 5-amino-4-oxopentanoic acid. 5-Aminolevulinic acid can be used as the salt, particularly a simple salt and especially the hydrochloride salt. 5-Aminolevulinic acid can also be used in the form of a precursor or product of 5-aminolevulinic acid. 5-Aminolevulinic acid can also be used in its pharmacologically equivalent form, such as an amide or ester. Examples of precursors and products of 5-aminolevulinic acid and pharmacologically equivalent forms of 5-aminolevulinic acid that can be used in the present invention are described in Kloek, et al. Prodrugs of 5-aminolevulinic acid for photodynamic therapy. Photochem Photobiol. 1996 December; 64(6):994-1000; WO 95/07077; Peng, et al., Build-up of esterified aminolevulinic-acid-derivative-induced porphyrin fluorescence in normal mouse skin, J Photochem Photobiol B. 1996 June; 34(1):95-6; and WO 94/06424. As used herein, all of these compounds, unless other wise noted, are referred to jointly and severally as “ALA.”

As used herein, “ALAS”, or “5-aminolevulinate synthase” is an enzyme known in the art by synthesizing 5-aminolevulinic acid and being critical to porphyrin production, and as described generally by Astner, et al. (Astner, et al. Crystal structure of 5-aminolevulinate synthase, the first enzyme of heme biosynthesis, and its link to XLSA in humans. EMBO J. 2005 Sep. 21; 24(18):3166-77. Epub 2005 Aug. 25.); “AONS” or “8-amino-7-oxononanoate synthase” is an acyltransferase known in the art involved in biotin synthesis; “CoA” or “coenzyme A” is a coenzyme known in the art for its use in the synthesis and oxidation of fatty acids. The specification also makes note of compounds and methods known in the art, such as “CD” or “circular dichroism”; “DEAE” or diethylaminoethyl; “HEPES” or “(N-[2-hydroxyethyl]piperazine-N′-[2-ethane sulfonic acid])”; “mALAS-2”, murine erythroid ALAS″; “MOPS” or “4-Morpholinepropanesulfonic acid”; “NAD⁺” or “β-nicotinamide adenine dinucleotide”; “PLP” or “pyridoxal 5′-phosphate; “SDS-PAGE” or “sodium dodecyl sulfate polyacrylamide gel electrophoresis”; “SS2” or “synthetically shuffled variant #2”.

DEAE-Sephacel, β-mercaptoethanol, PLP, bovine serum albumin, succinyl-CoA, ALA-hydrochloride, α-keto-glutarate, α-ketoglutarate dehydrogenase, Bis-Tris, HEPES-free acid, MOPS, thiamin pyrophosphate, NAD⁺, and the bicinchoninic acid protein determination kit were purchased from Sigma-Aldrich Chemical Company. Ultrogel AcA 44 was from IBF Biotechnics. Glycerol, glycine, disodium ethylenediamine tetraacetic acid dihydrate, ammonium sulfate, ascorbic acid and magnesium chloride hexahydrate were acquired from Fisher Scientific. Sodium dodecyl sulfate polyacrylamide gel electrophoresis reagents were acquired from Bio-Rad. PD-10 columns were from Amersham Biosciences. Restriction enzymes and polymerases were from New England Biolabs. Synthetic oligonucleotides were obtained from Integrated DNA Technologies.

As used herein, the term “hyperactive” refers to enzymes having the ability to enable at least a 20% increase in product formation compared to wild type enzymes at the same concentration and in the same environment. For example, a hyperactive ALAS enzyme includes one or more point mutations in the active site loop, thereby permitting the ALAS enzyme to yield approximately a kₐₜ increase of between increase 20% and 100%.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manuel (2D ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Based on the results of pre-steady state kinetic analyses of ALAS, a model wherein the enzyme exists in alternate “open” and “closed” conformations has been developed. (Hunter, & Ferreira. Pre-steady-state reaction of 5-aminolevulinate synthase. Evidence for a rate-determining product release J Biol. Chem. 1999 Apr. 30; 274(18):12222-8) An extended conformation “lid” near the C-terminus closes over the active site in the product bound structure. It was postulated that the product was sterically precluded from dissociating from the enzyme surface until this lid relaxes to the “open” position (Webster, et al. Mechanism of 8-amino-7-oxononanoate synthase: spectroscopic, kinetic, and crystallographic studies. Biochemistry. 2000 Jan. 25; 39(3):516-28; Alexeev, et al. The crystal structure of 8-amino-7-oxononanoate synthase: a bacterial PLP-dependent, acyl-CoA-condensing enzyme J Mol Biol. 1998 Nov. 27; 284(2):401-19), and that this conversion of the “closed” conformation to the “open” conformation, which is coincident with release of ALA from the enzyme surface, resulted in the rate-limiting step controlling the production of ALA from glycine and succinyl-CoA.

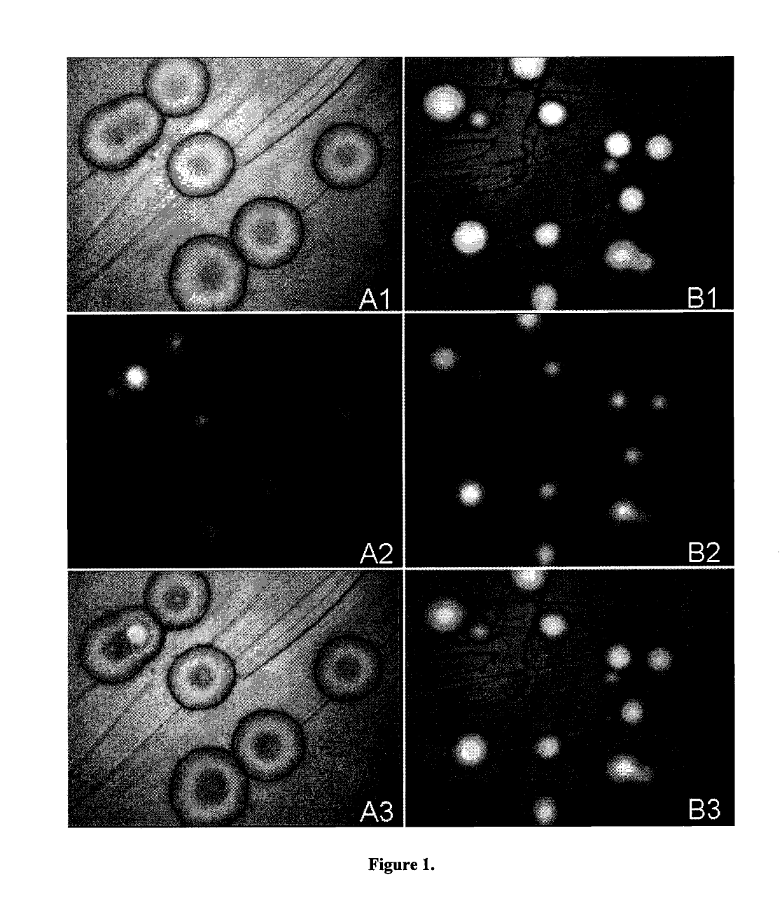

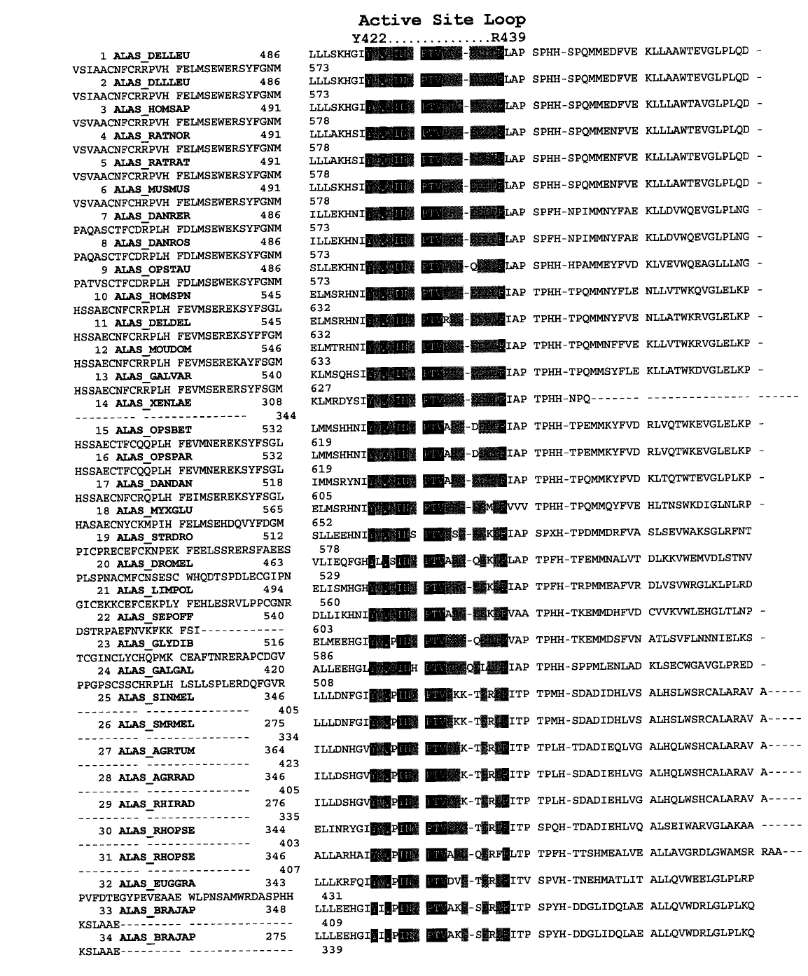

Further evidence that domain movement limits enzyme turnover comes from viscosity studies using glycerol as the viscogen, as seen in FIGS. 1(A1) through (B3). A linear correlation was found to exist between the maximal rate of reaction and solvent viscosity. The best fit line gives a slope statistically indistinguishable from one, indicating the reaction is 100% diffusion limited.

A mock vector was constructed with pTL26 plasmid, by replacing the murine, erythroid ALAS active site loop-encoding sequence with a non-ALAS-encoding sequence. pGF42, a ferrochelatase expression plasmid (Ferreira. Mammalian ferrochelatase. Overexpression in Escherichia coli as a soluble protein, purification and characterization. J Biol. Chem. 1994 Feb. 11; 269(6):4396-400), was digested with Xba I and Bam HI and the generated fragment was ligated into the ALAS expression plasmid, pGF23 (Ferreira, & Dailey, Expression of mammalian 5-aminolevulinate synthase in Escherichia coli. Overproduction, purification, and characterization. J Biol. Chem. 1993 Jan. 5; 268(1):584-90), previously digested with the same two enzymes.

The construction of the synthetic shuffled library depended on the design of five overlapping partially overlapping oligonucleotides, which contain DNA degeneracies to allow multiple mutations to be introduced in 10 of the 18 positions of the ALAS active site loop, seen in Table 1. The primers used in this study were the following (positions with base changes are indicated in bold):

(SEQ ID 1) Fwd Primer MALAS216:
5′-CTG CTC TCC AAG CAC AGC ATC TAT VTK CAG VSS ATC
AAC-3′
(SEQ ID 2) Rev Primer RALAS217:
5′-GGG GGC CAA GCG MAR BHK CWS VBB WYY NHB VBB CAC
AGT TGG RHD GTT GAT SSB CTG-3′
(SEQ ID 3) Fwd Primer MALAS218:
5′-CGC TTG GCC CCC TCC CCC CAC CAC AGC CCT CAG ATG
ATG GAA AAC TTT-3′
(SEQ ID 4) Fwd Primer MALAS219:
5′-ATC TGT GAT CTT CTG CTC TCC AAG CAC AGC AT-3′
(SEQ ID 5) Rev Primer RALAS220:
5′-CTT CTC CAC AAA GTT TTC CAT CAT CTG A-3′

where R=50% A+50% G; Y=50% C+50% T; M=50% A+50% C; K=50% G+50% T; S=50% C+50% G; W=50% A+50% T; H=A+25% C,T; B=33% C+33% G+33% T; V=33% A+33% C+33% G; D=33% A+33% G+33% T; N=25% A+25% C+25% G+25% T.

An annealing reaction (20 μL) containing 100 pmol of each of the five oligonucleotides was incubated at 65° C. for 2 min and then slowly cooled to room temperature. The annealed oligonucleotides were extended with 2 units of thermostable DNA polymerase and dNTPs, at final concentration of 55 μM each, in a 20 min reaction at room temperature.

The generated double-stranded DNA was used as template in a PCR reaction using the outermost forward and reverse primers (MALAS219 and RALAS220) and following the cycling parameters: a total of 30 cycles of 94° C. for 1 minute, 55° C. for 1 minute, and 72° C. for 1 minute was followed by an extension of 5 minutes at 72° C. The shuffled DNA product was further amplified by PCR and using 2 primers (MALAS166 and RpBRevo2—sequence below), which annealed to the 5′ and 3′ ends of the shuffled DNA product. The annealing sites for MALAS166 and RpBRevo2 were upstream and downstream of the sequences for the restriction endonucleases, Xba I and Bam HI, respectively. PCRs were performed using a MJ Research Minicycler and following the cycling parameters: a total of 30 cycles of 94° C. for 1 minute, 55° C. for 1 minute, and 72° C. for 1 minute was followed by an extension of 5 minutes at 72° C. The amplified, shuffled DNA product was digested with Xba I and Bam HI, purified and ligated into pTL26, the mock ALAS expression vector, previously digested with the same two enzymes, as described in (Gong & Ferreira. Aminolevulinate synthase: functionally important residues at a glycine loop, a putative pyridoxal phosphate cofactor-binding site. Biochemistry. 1995 Feb. 7; 34(5):1678-85).

| | |
|---|---|
| (SEQ ID 6) Fwd Primer MALAS166: | 5′-TGC AGG CCA TAG |
| | AGG AGA CC-3′ |
| (SEQ ID 7) Rev Primer RpBRevo2: | 5′-TAC GAG TTG CAT |
| | GAT AA-3′ |

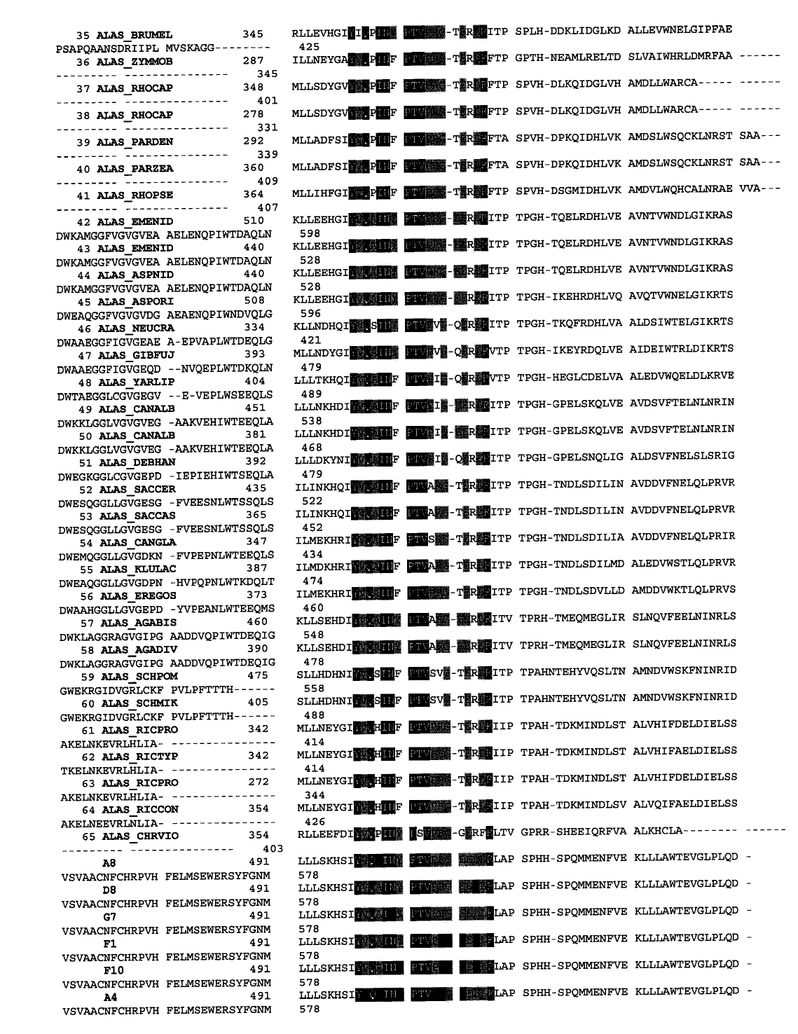

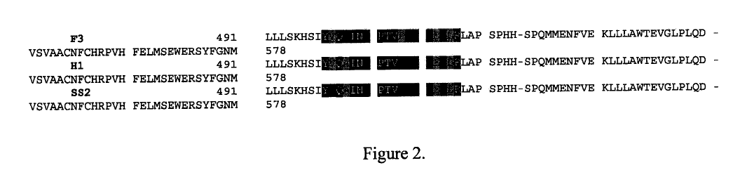

The design and experimental approach for construction of the library was based on previously described methods for shuffling mutagenesis (Ness, et al. Synthetic shuffling expands functional protein diversity by allowing amino acids to recombine independently. Nat Biotechnol. 2002 December; 20(12):1251-5. Epub 2002 Nov. 11; Ostermeier. Synthetic gene libraries: in search of the optimal diversity Trends Biotechnol. 2003 June; 21(6):244-7; Zha, et al. Assembly of designed oligonucleotides as an efficient method for gene recombination: a new tool in directed evolution. Chembiochem. 2003 Jan. 3; 4(1):34-9). The targeted region corresponded to mALAS-2 amino acids Y422-R439, as seen in FIG. 2. Codons for the ten non-conserved positions of the loop were targeted for shuffling mutagenesis using synthetic oligonucleotides as delineated in Table 1 and described in above.

TABLE 1
Designed mutations for incorporation at indicated
positions within the ALAS active site loop.¹

| Position | Wt | Mut1 | Mut2 | Mut3 | Mut 4 | Codon |
|---|---|---|---|---|---|---|
| 423 | V | I | L | | | VTK |
| 425 | A | S | P | | | VSS |
| 428 | Y | F | H | S | | HDY |
| 432 | P | A | S | D | R | VVB |
| 433 | R | I | V | K | S | VDN |
| 434 | G | K | | | | RRW |
| 435 | E | Q | T | S | D | VVB |
| 436 | E | L | | | | SWG |
| 437 | L | R | K | M | | MDV |
| 438 | L | | F | | | YTK |

¹WT denotes amino acid found in mALAS-2 active site loop. Amino acids indicated in the columns labeled Mut1-Mut4 reflect all naturally occurring amino acids found in the ALAS active site loop. Codon indicates the nucleotide codon used to obtain the indicated mixture of amino acid residues. DNA degeneracies are represented in the IUB code: Y, C/T; M, A/C; K, G/T; R, G/A; S, C/G; W, A/T; V, A/C/G; H, A/C/T; D, A/G/T; B, C/G/T; N, A/C/G/T.

The ALAS active site library was generated by annealing three partially overlapping oligonucleotides (1 pmol each) and amplifying the annealed product with PCR. The reaction product was further amplified with another round of PCR using primers overlapping the 5′ and 3′ ends of the generated PCR product and extending to the restriction enzyme sites used in subsequent subcloning (i.e., Xba I and Bam HI). To minimize the wild-type ALAS “background” during the screening of the library for functional ALAS variants, the library was subcloned into a mock ALAS expression vector. The mock vector contained the wild-type ALAS-encoding sequence from the pGF23 expression plasmid (Ferreira & Dailey. Expression of mammalian 5-aminolevulinate synthase in Escherichia coli. Overproduction, purification, and characterization. J Biol Chem. 1993 Jan. 5; 268(1):584-90), with the exception of the region encoding the active site loop and flanked by the Xba I and Bam HI restriction enzyme sites, which was replaced with a non-ALAS related sequence. The primers, conditions for the annealing reaction and PCR, and mock vector used in this study are described above. The ligation reactions and DNA digestions with restriction endonucleases were according to standard protocols in molecular biology.

### Example 1

### Screening of the ALAS Synthetic Library and Isolation of Functional ALAS Variants

A multiple sequence alignment of sixty-five different ALAS proteins was compiled with ClustalW software in order to determine the naturally occurring amino acid diversity present at specific positions in the active site loop, as seen in FIG. 2. This diversity was incorporated into mixed base oligonucleotide primers which were utilized to construct a library of theoretically over 10⁸ active site loop variants.

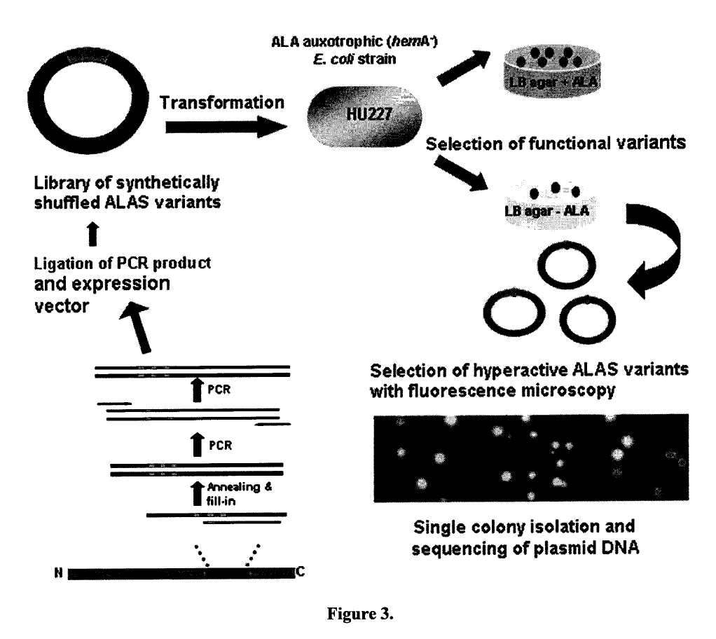

A simple and effective two-step library screening strategy was developed for isolation of colonies potentially harboring hyperactive ALAS variants, as seen in FIG. 3. First, the library was transformed into the ALA auxotrophic E. coli strain HU227 (hemA⁻) in order to eliminate non-active and very poorly active library variants (Gong & Ferreira. Aminolevulinate synthase: functionally important residues at a glycine loop, a putative pyridoxal phosphate cofactor-binding site. Biochemistry. 1995 Feb. 7; 34(5):1678-85; Li, et al. A heme-deficient strain of Escherichia coli has a three-base pair deletion in a “hotspot” in hemA. Biochim Biophys Acta. 2003 Apr. 15; 1626(1-3):102-5). Electrocompetent E. coli HU227 cells were transformed with the library and plated onto LB/ampicillin medium without ALA to allow selection of the active ALAS variants as previously described (Gong & Ferreira. Aminolevulinate synthase: functionally important residues at a glycine loop, a putative pyridoxal phosphate cofactor-binding site. Biochemistry. 1995 Feb. 7; 34(5):1678-85), as seen in FIG. 3.

To score the total number of colonies produced and assess transformation efficiency, one-tenth of each transformation reaction was spread onto LB/ampicillin plates containing 10 μg/ml ALA. Functional ALAS clones (i.e., isolated from the ALA minus plates) were then picked and plated onto defined MOPS medium to induce protein overexpression (Ferreira & Dailey. Expression of mammalian 5-aminolevulinate synthase in Escherichia coli. Overproduction, purification, and characterization. J Biol Chem. 1993 Jan. 5; 268(1):584-90) and screened for porphyrin overproduction using fluorescence microscopy. Briefly, the plates with the functional ALAS clones were examined with a Nikon Eclipse E1000 fluorescence microscope (Nikon, Tokyo) fitted with either a Nikon Triple Band filter set for excitation at 385-400 nm and emission at 450-465 nm for 4′,6-diamidino-2-phenylindole (DAPI) detection or a Nikon Cube BV2A excitation filter set for excitation at 400-440 nm and emission at 450-465 nm with a 610 nm long pass filter and a band pass filter at 550 nm±20 nm for porphyrin detection. Photographs were taken with a CCIR high performance COHU CCD camera and the images were processed with Image software Genus 2.81 from Applied Imaging.

Next, the intensity of porphyrin fluorescence following induction of ALAS expression was used as a presumptive indicator of relative activity. Porphyrin fluorescence accumulated in the functional ALAS clones was compared to that of bacterial cells harboring wild-type ALAS and grown under the same experimental conditions. The porphyrin fluorescence in individual colonies differed substantially, as exemplified in FIG. 1, (Ferreira, & Dailey, Expression of mammalian 5-aminolevulinate synthase in Escherichia coli. Overproduction, purification, and characterization. J Biol Chem. 1993 Jan. 5; 268(1):584-90).

### Qualitative Analysis of the Isolated Active Site Loop Variants.

Colonies accumulating greater porphyrin fluorescence density than those harboring wild-type ALAS were individually grown overnight in LB/ampicillin medium in 96-well plates, and glycerol stocks generated from the cultures were submitted to the ICBR Genomics Core at the University of Florida for DNA sequencing of the corresponding plasmids.

1820 of 3800 screened colonies rescued growth of HU227 E. coli cells. Plasmid DNA from 194 colonies producing high porphyrin concentrations was sequenced in the region encompassing the active site loop, and confirmed the integrity of the library (data not shown). Nine independent ALAS variants from bacterial cells producing exceptionally high porphyrin concentrations were purified and characterized. Sequence data for this subset indicated that functional mutations were present at eight of the ten positions targeted, the exceptions being Glu-436 and Leu-438, as seen in Table 2.

TABLE 2
Amino acids substitutions in hyperactive mALAS-2
variants. Targeted positions include changes
observed in other ALASs and mutations not
observed in nature.

| | | | | | | | | | | | | | | | | | | |
|---|---|---|---|---|---|---|---|---|---|---|---|---|---|---|---|---|---|---|
| Wild-type ALAS | Y422 | V | Q | A | I | N | Y | P | T | V | P | R | G | E | E | L | L | R439 |
| (SEQ ID 8) | | | | | | | | | | | | | | | | | | |
| Single | | | | | | | | | | | | | | | | | | |
| variants | | | | | | | | | | | | | | | | | | |

| | | | | | | | | | | | |
|---|---|---|---|---|---|---|---|---|---|---|---|
| (SEQ ID 9) | A8 | | T | | | | | | | | |
| (SEQ ID 10) | D8 | | | | | | | | | | Q |
| (SEQ ID 11) | G7 | | | H | | | | | | | |
| Quad | | | | | | | | | | | |
| variant | | | | | | | | | | | |
| (SEQ ID 12) | F1 | | | | | | K | K | Q | | Q |
| Penta | | | | | | | | | | | |
| variant | | | | | | | | | | | |
| (SEQ ID 13) | F10 | | | | I | | Q | N | T | | N |
| Hexa | | | | | | | | | | | |
| variants | | | | | | | | | | | |
| (SEQ ID 14) | A4 | I | P | | C | R | K | | N | | |
| (SEQ ID 15) | F3 | | G | | H | H | N | K | | K | |
| (SEQ ID 16) | H1 | | | | N | N | I | E | K | | K |
| Hepta | | | | | | | | | | | |
| variant | | | | | | | | | | | |
| (SEQ ID 17) | SS2 | L | | | R | E | I | N | Q | | K |

Colonies were visualized with a DAPI filter in panels A1 and B1, by red fluorescence in panels A2 and B2, and as overlays in panels A3 and B3. A decrease in colony diameter was also observed to be a general feature of bacterial cells harboring hyperactive ALAS variants, presumably due to oxidative toxicity associated with overproduction of ALA (Hunter, et al. Supraphysiological concentrations of 5-aminolevulinic acid dimerize in solution to produce superoxide radical anions via a protonated dihydropyrazine intermediate. Arch Biochem Biophys. 2005 May 15; 437(2):128-37. Epub 2005 Mar. 22). Thus, porphyrin fluorescence intensity proved to be a reliable indicator of ALAS activity.

### Example 2

### Overexpression, Purification and Spectroscopic Analyses of ALAS Active Site Loop Variants

Overexpression was from the alkaline phosphatase (phoA) promoter, and the conditions for promoter induction were as previously described for mALAS-2 (Ferreira, & Dailey, Expression of mammalian 5-aminolevulinate synthase in Escherichia coli. Overproduction, purification, and characterization. J Biol Chem. 1993 Jan. 5; 268(1):584-90). However, induction of the F1, SS2, F10 and H1 variants was accomplished by growing the bacterial cells harboring the expression plasmids for these variants in MOPS medium supplemented with 10 mg/L ascorbic acid for 30 hours at 20° C., as these conditions ameliorated the apparent toxicity observed with overexpression of the most active variants, and produced better yields. Purification, storage, handling, and spectroscopic analysis of the mALAS-2 variants were conducted as described previously (Hunter, & Ferreira. Pre-steady-state reaction of 5-aminolevulinate synthase. Evidence for a rate-determining product release J Biol Chem. 1999 Apr. 30; 274(18):12222-8). Protein concentrations were determined by the bicinchoninic acid method using bovine serum albumin as the standard (Ferreira, & Dailey, Expression of mammalian 5-aminolevulinate synthase in Escherichia coli. Overproduction, purification, and characterization. J Biol Chem. 1993 Jan. 5; 268(1):584-90). Reported enzyme concentrations are based on the monomeric molecular mass of 56 kDa (Ferreira, & Dailey, Expression of mammalian 5-aminolevulinate synthase in Escherichia coli. Overproduction, purification, and characterization. J Biol Chem. 1993 Jan. 5; 268(1):584-90). Protein purity was assessed using SDS-PAGE.

### Steady-State and Pre-Steady-State Kinetic Characterization of ALAS Active Site Loop Variants.

ALAS steady-state activity of the ALAS active site loop variants was determined at 20° C. using a continuous spectrophotometric assay as described previously for wild-type ALAS (Hunter & Ferreira. A continuous spectrophotometric assay for 5-aminolevulinate synthase that utilizes substrate cycling. Anal Biochem. 1995 Apr. 10; 226(2):221-4). Rapid scanning stopped-flow measurements were performed using a model RSM-100 stopped-flow spectrophotometer (OLIS, Inc.). This instrument has a dead-time of approximately 2-ms and an observation chamber path length of 4 mm Spectral scans covering a wavelength range of 300-570 nm were collected at a rate of 1000 scans/s and then averaged to 62 scans/s in order to reduce the time course data files to an appropriate size for global fit analyses. The temperature of the syringes and the stopped-flow cell compartment was maintained at 20° C. by an external water bath. Pre-steady-state kinetic reactions of the variant enzymes were examined under single turnover conditions, using final concentrations of 60 μM enzyme, 120 mM glycine and 10 μM succinyl-CoA in 100 mM HEPES, pH 7.5 and 10% (v/v) glycerol as previously described (Hunter, & Ferreira. Pre-steady-state reaction of 5-aminolevulinate synthase. Evidence for a rate-determining product release J Biol Chem. 1999 Apr. 30; 274(18):12222-8). Single turnover data were evaluated using either a two- or three-kinetic-step mechanism as described by Equations 1 and 2, respectively (Hunter, & Ferreira. Pre-steady-state reaction of 5-aminolevulinate synthase. Evidence for a rate-determining product release J Biol Chem. 1999 Apr. 30; 274(18):12222-8).

$$\begin{matrix}A {}^{{k}_{obs}^{1}} B {}^{{k}_{obs}^{2}} C & ( Eq . 1 ) \\ A {}^{{k}_{obs}^{1}} B {}^{{k}_{obs}^{2}} C {}^{{k}_{obs}^{3}} D & ( Eq . 2 )\end{matrix}$$

The spectral data was used to determine the observed rate constants via the Robust Global Fitting program. This method utilizes the single value decomposition software provided by OLIS, Inc. as previously reported (Hunter, & Ferreira. Pre-steady-state reaction of 5-aminolevulinate synthase. Evidence for a rate-determining product release J Biol Chem. 1999 Apr. 30; 274(18):12222-8; Hunter, et al. Transient kinetic studies support refinements to the chemical and kinetic mechanisms of aminolevulinate synthase. J Biol Chem. 2007 Aug. 10; 282(32):23025-35. Epub 2007 May 7). Quality of the calculated fits was judged by analysis of the residuals, and the simplest mechanism that described the experimental data was used.

Activity data were acquired using a Shimadzu UV/Vis 2100 dual-beam spectrophotometer, and are summarized in Table 3. These data were used to calculate steady-state kinetic parameters using non-linear regression analysis software (SigmaPlot10, Systat, CA). The ALAS steady-state kinetic parameters of wild-type ALAS and the SS2 variant activity assays were also determined at 15, 25, and 35° C.

TABLE 3
Kinetic parameters for the reactions of hyperactive ALAS enzymes¹

| | Steady-state parameters | | | | | Pre-steady-state parameters | | |
|---|---|---|---|---|---|---|---|---|
| | K_{cat} | Kₘ^{Gly} | Kₘ^{SCoA} | K_{cat}/Kₘ^{Gly} | K_{cat}/Kₘ^{Gly} | Q_{f}² | Q_{d1}³ | Q_{d2}⁴ |
| Enzyme | s⁻¹ | mM | μM | mM · s⁻¹ | μM · s⁻¹ | s⁻¹ | s⁻¹ | s⁻¹ |
| WT | 0.02 ± 0.01 | 14 ± 2.0 | 11 ± 1.0 | 1.1 × 10⁻³ | 1.8 × 10⁻³ | 0.8 | 0.53 | 0.01 |
| SS2 | 0.31 ± 0.06 | 12 ± 1.2 | 3.0 ± 0.3 | 0.02 | 0.10 | 16 | 1.7 | N/A |
| A4 | 0.16 ± 0.01 | 14 ± 1.1 | 2.3 ± 0.4 | 0.01 | 0.07 | 7.1 | 1.7 | 0.07 |
| F3 | 0.20 ± 0.01 | 13 ± 1.1 | 2.6 ± 0.4 | 0.02 | 0.08 | 7.7 | 1.1 | 0.17 |
| H1 | 0.23 ± 0.14 | 13 ± 1.4 | 2.0 ± 0.3 | 0.02 | 0.11 | 10 | 1.5 | N/A |
| F10 | 0.17 ± 0.01 | 16 ± 1.8 | 1.1 ± 0.7 | 0.01 | 0.15 | 3.7 | 0.98 | 0.15 |
| F1 | 0.20 ± 0.02 | 16 ± 1.2 | 2.9 ± 0.4 | 0.01 | 0.07 | 4.0 | 1.1 | N/A |
| A8 | 0.07 ± 0.01 | 25 ± 3.7 | 2.3 ± 0.4 | 2.8 × 10⁻³ | 0.03 | 4.8 | 0.81 | 0.12 |
| D8 | 0.17 ± 0.01 | 24 ± 3.1 | 1.7 ± 0.7 | 1.3 × 10⁻³ | 0.18 | 5.4 | 0.44 | 0.02 |
| G7 | 0.17 ± 0.01 | 15 ± 1.7 | 1.5 ± 0.1 | 4.6 × 10⁻³ | 0.05 | 4.4 | 0.87 | 0.12 |

¹Enzymatic reactions monitored at 20° C.
²Rate for quinonoid intermediate formation.
³Rate for first step of quinonoid intermediate decay.
⁴Rate for second step of quinonoid intermediate decay.

Screening a small subset of the active site loop library resulted in isolation of nine different hyperactive ALASs. Single-turnover reactions were utilized to determine the kinetics of quinonoid intermediate formation and decay. Turnover numbers and catalytic efficiencies were increased for each of the purified variant enzymes, as seen in Table 3. Hyperactivity was arbitrarily defined as at least a ten-fold increase in catalytic efficiency towards one or both substrates, and each of the purified variant enzymes met this criterion. The catalytic efficiencies for succinyl-CoA were increased up to one hundred-fold, while those for glycine were increased in proportion to increases in k_{cat}, as the Kₘ values for glycine were either unchanged or slightly elevated.

The catalytic efficiencies for succinyl-CoA were enhanced to a greater extent than those for glycine, consistent with the role of the active site loop in binding of this substrate. The variety of sequence alterations leading to hyperactivity suggests that ALAS is not evolving towards catalytic perfection, and probably has evolved towards a highly controlled rate of ALA production consistent with its role in control of porphyrin biosynthesis. It also suggests the possibility of one or more as yet undiscovered allosteric effectors or post-translational modifications that modulate ALAS activity in vivo.

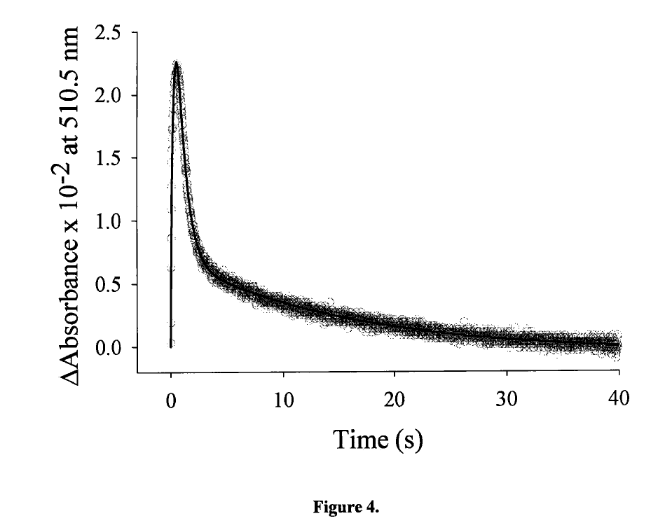

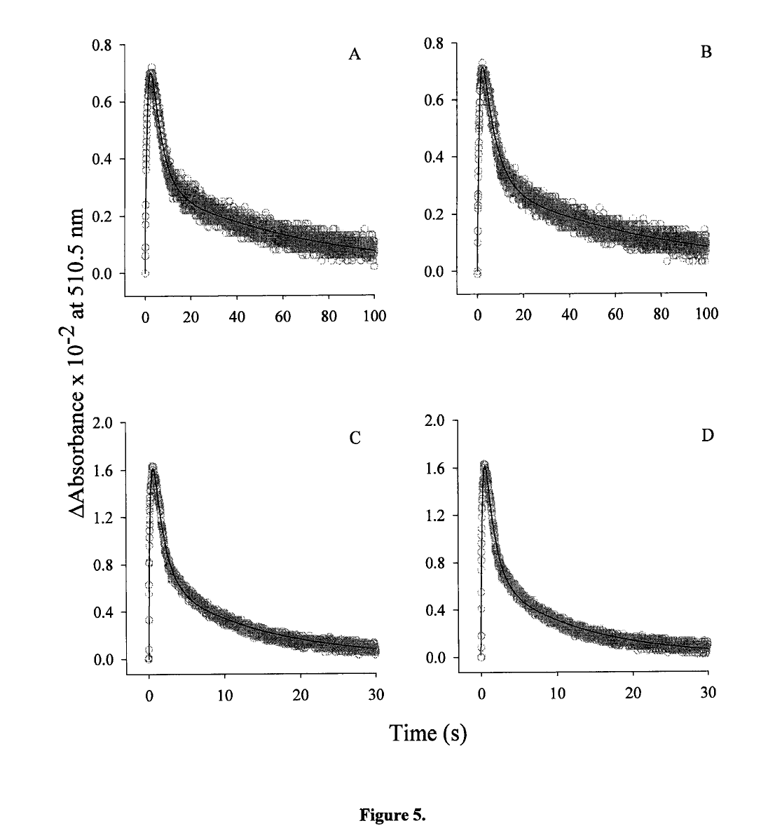

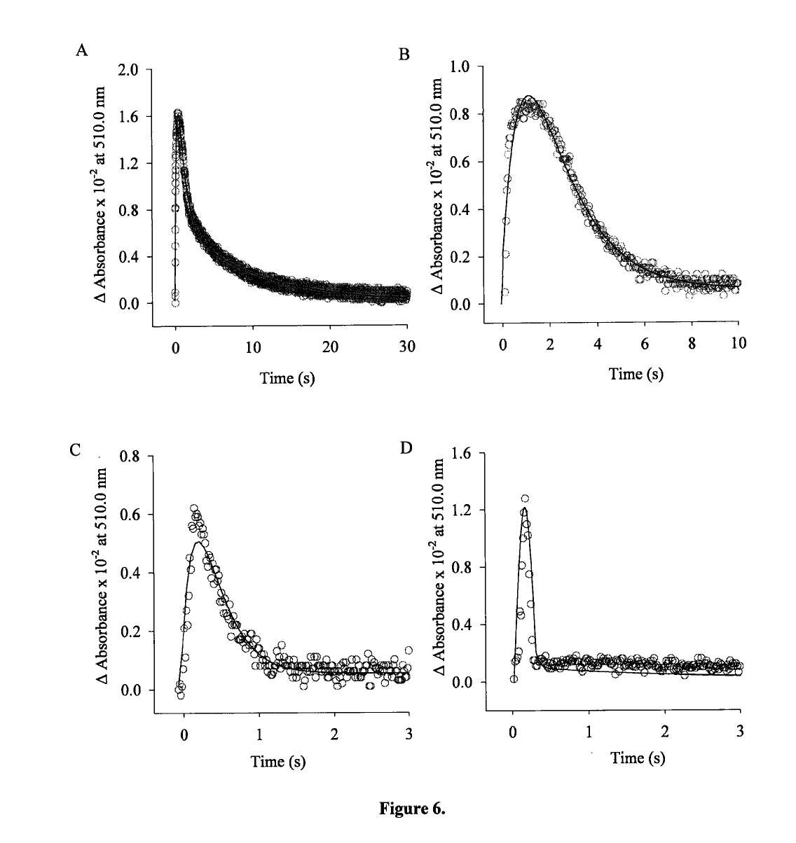

In the wild-type enzyme single-turnovers occur in three steps, as seen in FIG. 4, which have been assigned to decarboxylation of a PLP-bound α-amino-β-ketoadipate intermediate to form a PLP-ALA quinonoid intermediate; protonation of the quinonoid intermediate to form the bound product; and opening of the active site loop coincident with product released (Hunter, et al. Transient kinetic studies support refinements to the chemical and kinetic mechanisms of aminolevulinate synthase. J Biol Chem. 2007 Aug. 10; 282(32):23025-35. Epub 2007 May 7). Quinonoid intermediate formation for the A4 and F3 hexa-variants were increased by nine-fold (7.1 s⁻¹ and 7.7 s⁻¹, respectively) compared to wild-type ALAS (0.8 s⁻¹). Additionally, the rates corresponding to the first step of quinonoid intermediate decay in these variants (1.7 s⁻¹ and 1.1 s⁻¹) were also increased over that of wild-type ALAS (0.53 s⁻¹). Similarly, the second step of quinonoid intermediate decay, a step hypothesized to include opening of the active site loop and ALA dissociation, was markedly increased in these variants, with values of 0.07 s⁻¹ and 0.17 s⁻¹, rates 7- and 17-fold higher when compared to 0.01 s⁻¹ for wild-type ALAS. These data support the increased catalytic efficiency observed from the experiments performed in the steady-state. The singly mutated variants A8, D8 and G7, along with A4, F3 and F10, formed the quinonoid intermediate at least 4-fold faster when compared to wild-type ALAS (4.8 s⁻¹, 5.4 s⁻¹, 4.4 s⁻¹, respectively), as seen in FIGS. 5(A) through 5(D), 6(A) and 6(B). However, the first rate of biphasic quinonoid intermediate decay was similar to that of wild-type ALAS. In the remaining 3 variants, the lifetime of the transient quinonoid intermediate was dramatically different, as seen in FIGS. 6(C), 6(D) and 7. Instead of proceeding by a mechanism similar to wild-type ALAS, these enzymes appear to condense the biphasic rate of decay into a single step. Consequently, the data are fitted to a two step sequential mechanism.

The most logical interpretation of the absence of a second step of quinonoid intermediate decay in the reactions of the SS2, H1, and F1 variants is that this step has become faster than the first step of quinonoid intermediate decay and is thus kinetically insignificant. This would imply a fundamental change in the nature of the rate-determining step of the reaction cycle in these variants, such that opening of the active site loop coincident with ALA release is no longer the slowest step. The steady-state temperature dependence studies with the SS2 variant support this conclusion, as the Q₁₀ for the catalytic rate constant is reduced from seven to two. From the single-turnover rates it can be inferred that in the SS2 variant-catalyzed reaction product release has been accelerated by at least 170-fold (Q_{d2}^{SS2} must be ≧Q_{d1}^{SS2}, which is in turn 170-fold greater than Q_{d2}^{wt}). SS2, H1, and F1 each form the quinonoid intermediate faster than wild-type ALAS. Most notably, SS2 does so at a rate that is 20-fold faster (16 s⁻¹ vs. 0.8 s⁻¹ for wild-type ALAS), as seen in FIG. 7. The rate associated with quinonoid intermediate decay was also enhanced in these variants.

### Determination of the Dissociation Constants for the Binding of Glycine and ALA.

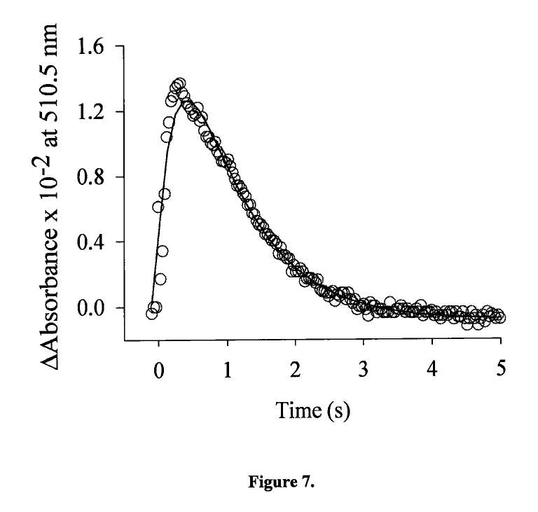

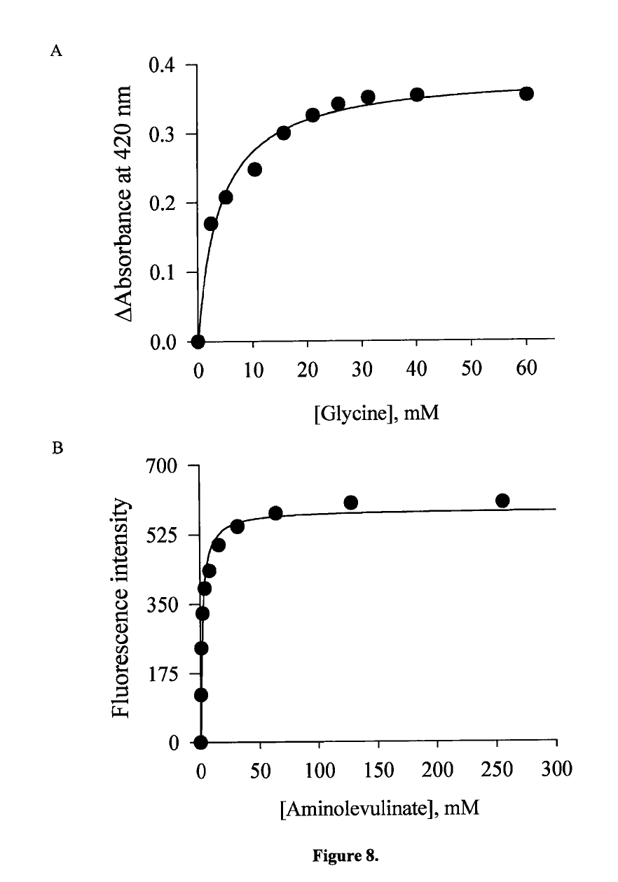

The equilibrium dissociation constant (K_{D}) for the binding of glycine to the SS2 variant was determined in 20 mM HEPES, pH 7.5, containing 10% glycerol at 20° C. and by titrating the SS2 variant (60 μM) with increasing concentrations of glycine (0.6 mM-60 mM), as seen in FIGS. 8(A) and 8(B). The reaction was monitored by the increase in absorbance at 420 nm upon formation of the external aldimine between PLP and glycine (Nandi. Studies on delta-aminolevulinic acid synthase of Rhodopseudomonas spheroides. Reversibility of the reaction, kinetic, spectral, and other studies related to the mechanism of action. J Biol Chem. 1978 Dec. 25; 253(24):8872-7). Schiff base formation between the cofactor of the SS2 variant and glycine was monitored spectroscopically at 420 nm While the holoenzyme-only UV-Vis absorbance spectra showed a modest decrease in the ratio of 330 nm/420 nm, when compared to that of wild-type ALAS (data not shown), the amplitudes at 420 nm corresponding to the glycine-bound enzymes were concentration dependent. The glycine concentration dependent data were fit to a standard hyperbola (Eq. 3) and the K_{D} value was determined by fitting the data to Equation 3, where ΔY is the absorbance increase at 420 nm, Yₘₐₓ is the maximum increase in absorbance, and [Ligand] is the glycine concentration, using non-linear regression analysis. Binding of ALA to the SS2 variant resulted in quenching of the fluorescence emission at 428 nm upon excitation at 330 nm due to formation of an external aldimine with the PLP-cofactor. Thus to determine the dissociation constant for the binding of ALA to SS2, the change in fluorescence emission at 428 nm (λ_{exc}=330 nm) was monitored upon titration of SS2 (60 μM) with increasing concentrations of ALA (0.5 mM-128 mM). The changes in fluorescence at 428 nm were plotted as a function of ALA concentration, and the K_{D} value was determined by fitting the data to Equation 3, using non-linear regression analysis software. In this application of Equation 3, ΔY is the total change in fluorescence at 428 nm, Yₘₐₓ is initial fluorescence, and [Ligand] is the ALA concentration.

$$Δ Y = \frac{{Y}_{max} [ Ligand ]}{{K}_{D} + [ Ligand ]} \qquad ( Eq . 3 )$$

K_{D} for glycine was found to be 4.12±0.57 mM, which is 50% lower than that of the wild-type enzyme. The increased affinity of SS2 for glycine suggests that the active site lid mutations facilitate substrate binding, a finding coincident with increased catalytic efficiency. Analysis of K_{D} for ALA indicated a dissociation constant of 1.82±0.19 mM, a value 62% higher compared to that of wild-type ALAS. This value is consistent with an increased rate of product release.

### Determination of the Thermodynamic Activation Parameters.

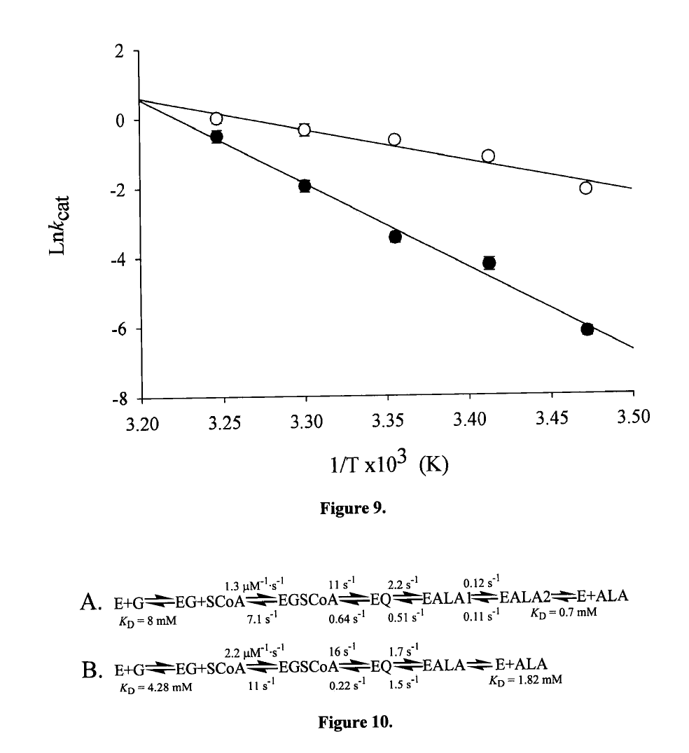

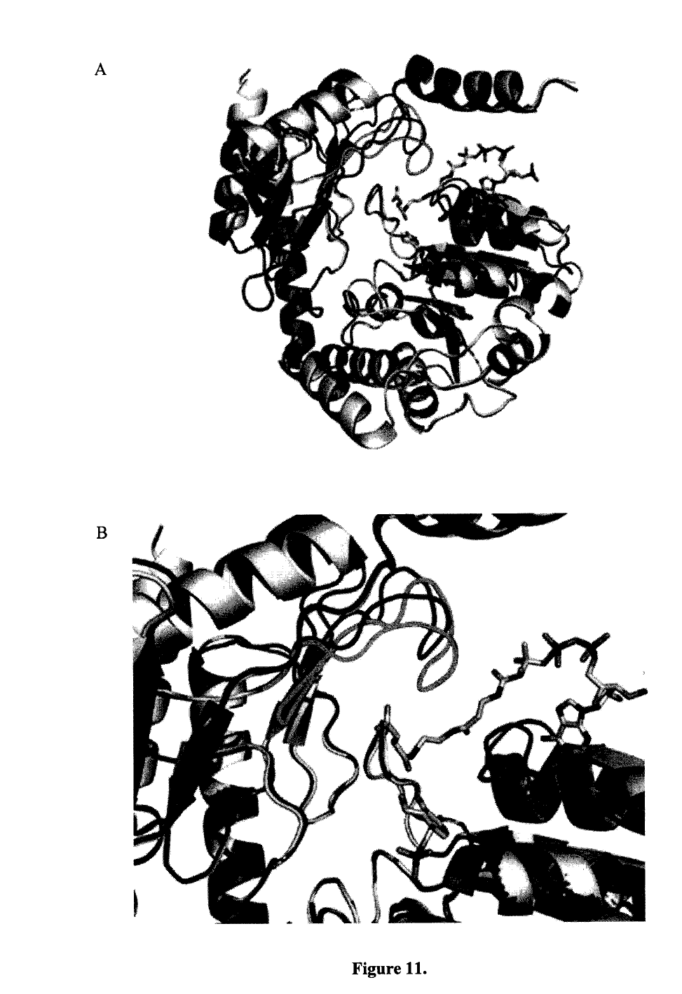

The temperature dependence of the steady-state kinetic parameters of wild-type ALAS and the SS2 variant were examined as described above the range 288-308 K for both wild-type ALAS and the SS2 variant, as seen in FIG. 9. The natural log of the calculated values for the turnover numbers (lnk_{cat}) were plotted vs. the inverse of temperature and the data were fit to the Arrhenius equation.

$$ln ( {k}_{obs} ) = \frac{- {E}_{a}}{R} \frac{1}{T} + ln ( A ) \qquad ( Eq . 4 )$$

where Eₐ is the activation energy, R is the universal gas constant, T is the absolute temperature, and A is the frequency factor. The determined activation energies were then used to calculate the thermodynamic activation parameters, ΔH, ΔG and ΔS, as previously described (Hunter, et al. Transient kinetic studies support refinements to the chemical and kinetic mechanisms of aminolevulinate synthase. J Biol. Chem. 2007 Aug. 10; 282(32):23025-35. Epub 2007 May 7). The turnover numbers for each enzyme were used to construct an Arrhenius plot, from which the thermodynamic activation parameters were derived seen in Table 4.

TABLE 4
Thermodynamic activation parameters of
wild-type ALAS and the SS2 variant.

| | | | ΔH^{‡} | ΔG^{‡} | ΔS^{‡} |
|---|---|---|---|---|---|
| | | Eₐ | (30° C.) | (30° C.) | (30° C.) |
| Enzyme | Slope/(K) | (kcal/mol) | (kcal/mol) | (kcal/mol) | (cal/mol · K) |
| SS2 | −9200 | 18 | 18 | 19 | −4.4 |
| WTALAS | −24000 | 48 | 48 | 19 | −95 |

KinTekSim kinetic simulation software (Barshop, et al. Analysis of numerical methods for computer simulation of kinetic processes: development of KINSIM—a flexible, portable system. Anal Biochem. 1983 Apr. 1; 130(1):134-45) was used to model the single wavelength kinetic traces at 510 nm for the reaction catalyzed by the SS2 variant and thus estimate forward and reverse rate constants. The interior rate constants were allowed to float, while the previously determined K_{D} values for binding of glycine and ALA to the SS2 variant and wild-type ALAS were held constant (Hunter, & Ferreira. Pre-steady-state reaction of 5-aminolevulinate synthase. Evidence for a rate-determining product release J Biol Chem. 1999 Apr. 30; 274(18):12222-8). The Arrhenius plot of the experimental data showed a linear dependence in the temperature range for both enzymes. From the slope of the straight line, an activation energy (Eₐ) of 0.48 kcal/mol was calculated for the wild-type enzyme. For the SS2 enzyme, Eₐ was determined to be 0.18 kcal/mol, a value 80% lower compared to that of wild-type ALAS. This suggests that the SS2-catalyzed reaction may decrease the energy barrier for enzyme motions that are coupled to the reaction. Different slope values were calculated from the linear relationship between the k_{cat} values for both enzymes and temperature. The slope value associated with wild-type ALAS was −24000, compared to −9200 for the SS2 variant. These two dissimilar values suggest that the SS2-catalyzed reaction proceeds by an alternate reaction pathway, where chemistry and lid mobility determines the rate-limiting step, as seen in FIG. 10.

A growing body of experimental evidence supports the theory that enzyme dynamics are not only integral to but also often synchronous with key kinetic steps during biocatalysis (Eisenmesser, et al. Intrinsic dynamics of an enzyme underlies catalysis. Nature. 2005 Nov. 3; 438(7064):117-21; Benkovic, et al. Free-energy landscape of enzyme catalysis. Biochemistry. 2008 Mar. 18; 47(11):3317-21. Epub 2008 Feb. 26). Product release is rate-determining for many enzymatic reactions, and in several cases where this step has been more closely examined, a conformational change of the enzyme has been discovered to be the key kinetic barrier (Hanson, J. A., et al. Illuminating the mechanistic roles of enzyme conformational dynamics. Proc Natl Acad Sci USA. 2007 Nov. 13; 104(46):18055-60. Epub 2007 Nov. 7; Rozovsky & McDermott. The time scale of the catalytic loop motion in triosephosphate isomerase. J Mol Biol. 2001 Jun. 29; 310(1):259-70; Boehr, et al. An NMR perspective on enzyme dynamics Chem Rev. 2006 August; 106(8):3055-79; Raber, et al. Dissection of the stepwise mechanism to beta-lactam formation and elucidation of a rate-determining conformational change in beta-lactam synthetase. (2009) J. Biol Chem. 2009 Jan. 2; 284(1):207-17. Epub 2008 Oct. 27).

The nature of the amino acid changes observed in the isolated hyperactive variants can in most cases be logically interpreted in terms of enzyme structure and function. For instance, the k_{cat} value has been reported to increase 20% and 100% for the R433L and R433K mALAS-2 variants, respectively (Tan, et al. Role of arginine 439 in substrate binding of 5-aminolevulinate synthase. Biochemistry. 1998 Feb. 10; 37(6):1478-84). The R. capsulatus crystal structures revealed that this loop residue comes within bifurcated hydrogen bonding distance of a conserved acidic residue (D81 in mALAS-2) in the closed conformation. Disruption of this electrostatic interaction would be expected to destabilize the closed conformation and provide a rationale for the observed increase in activity, and changes at this position were observed in two-thirds of the hyperactive variants isolated.

In general, the hyperactive loop variants harbor mutations that increase both hydrophilicity and basicity. These changes presumably destabilize and increase the “floppiness” of the loop by increasing solubility while eliminating hydrophobic and electrostatic interactions that would otherwise stabilize the loop in the closed conformation. Asparagine and glutamine would be expected to occur at an average rate of twelve percent at the codons potentially coding for these residues, based on the synthetic oligonucleotides used to prepare the library, as seen in Table 1, yet they are observed at twice this rate in the isolated hyperactive loop variants, as seen in Table 2. Similarly, lysine is expected to randomly occur, on average, seven percent of the time at the positions that include codons for it, but is observed in seventeen percent of the hyperactive variant positions potentially coding for it. The strongest selective pressure was observed for wild-type residues, however, as exemplified by a six-fold greater occurrence of proline at position 432 than would be expected based on the synthetic oligonucleotides used to prepare the library.

The accelerations observed for the pre-steady state rates associated with intermediate decarboxylation (quinonoid intermediate formation) and protonation of the ALA bound quinonoid intermediate (first step of quinonoid intermediate decay) indicate that non-conserved loop residues can have effects not only on the rate of product release, but also on multiple chemical steps in the catalytic cycle. It is thus reasonable to posit that loop dynamics are attuned with multiple steps in the reaction cycle.

In conclusion, the isolation and characterization of a variety of stable hyperactive ALAS variants lead us to propose that the loop is an important determinant in ALAS activity and appear to be a hotspot for generation of hyperactive variants. These and other ALA-overproducing ALAS variants may be useful in multiple clinical and agricultural applications. In fact, a number of these applications have been identified to date (Ziolkowski, et al. Enhancement of photodynamic therapy by use of aminolevulinic acid/glycolic acid drug mixture. J Exp Ther Oncol. 2004 July; 4(2):121-9; Wang, et al. Promotion of 5-aminolevulinic acid on photosynthesis of melon (Cucumis melo) seedlings under low light and chilling stress conditions. Physiol Plant. 2004 June; 121(2):258-264; Hua, et al. Effectiveness of delta-aminolevulinic acid-induced protoporphyrin as a photosensitizer for photodynamic therapy in vivo. Cancer Res. 1995 Apr. 15; 55(8):1723-31). The greatest of interest is tumor therapy by photosensitizing porphyrins, whereby the porphyrin drug is targeted to the tumor and irradiated to elicit the desired pharmacological response (e.g., apoptosis) (Dolmans, et al. Photodynamic therapy for cancer. Nat Rev Cancer. 2003 May; 3(5):380-7). These enzyme variants, which are defined by accelerated rates of turnover, could complement this technique by biosynthesizing the necessary porphyrin precursor molecules after localized delivery of their parent DNA to the tumor site.

In the preceding specification, all documents, acts, or information disclosed do not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a ALAS hyperactive enzyme, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. An ALAS enzyme comprising:
an enzymatic body;
an active site loop at amino acids 422 to 439, wherein the amino acids are N₁-Q-N₂-I-N₃-N₄-P-T-V-N₅-N₆-N₇-Ng-N₉-E-N₁₀-L;
where N₁ is selected from the amino acids V, I, and L;
where N₂ is selected from the amino acids A, T, P, and G;
where N₃ is selected from the amino acids N, and H;
where N₄ is selected from the amino acids Y, I, C, H, N, and R;
where N₅ is selected from the amino acids P, R, N, and E;
where N₆ is selected from the amino acids R, K, Q, H, and I;
where N₇ is selected from the amino acids G, K, N, and E;
where N₈ is selected from the amino acids E, Q, T, N, and K;
where N₉ is selected from the amino acids L, Q, N, and K; and
where N₁, N₂, N₃, N₄, N₅, N₆, N₇, N₈, N₉, N₁₀ are not simultaneously V, A, N, Y, P, R, G, E, L, respectively.

2. The ALAS enzyme of claim 1, wherein the active site loop amino acids are selected from the group consisting of SEQ ID No. 9, 10, 11, 12, 13, 14, 15, 16, and 17.

3. A method of generating an ALAS enzyme, comprising:
providing a nucleic acid sequence encoding for ALAS protein;
generating synthetically shuffled mutations at amino acids 422 to 439;
amplifying the annealed product;
introducing the annealed product into an expression vector;
screening the product for porphyrin overproduction, wherein the overproduction of porphyrin indicates the increase in product formation.

4. The method of claim 3, wherein the mutations are generated by annealing a plurality of partially overlapping oligonucleotides.

5. The method of claim 3, wherein the mutations are introduced with shuffling mutagenesis.

6. The method of claim 3, wherein the porphyrin overproduction is screened using fluorescence microscopy.

7. The method of claim 3, wherein the fluorescence microscopy is conducted at an excitation wavelength selected from the range of 385-400 nm and 400-440 nm;
and wherein the microscopy screening is conducted at an emission wavelength of 450-465 nm.

8. The method of claim 3, further comprising subcloning a DNA fragment encoding the synthetically shuffled mutations at amino acids 422 to 439, wherein the subcloning is performed by:
ligating the DNA fragment encoding the synthetically shuffled mutations at amino acids 422 to 439 into an expression vector by introducing selective pressure.

9. The method of claim 8, wherein the selective pressure is removal of ALA from the growth medium.

10. The method of claim 8, wherein the bacteria is grown in a medium of MOPS medium supplemented with ascorbic acid.

11. The method of claim 8, wherein the bacteria is Escherichia coli.

12. The method of claim 11, wherein the selective Escherichia coli is auxotrophic for ALA.

13. The method of claim 3, further comprising characterizing the ALAS variants, wherein the characterization is performed by:
measuring the equilibrium dissociation constant, further comprising
monitoring absorbance at 420 nm; and
calculating the dissociation constant using non-linear regression analysis.

14. The method of claim 3, further comprising screening the library by
transforming microbes with an ALAS variant-encoding expression plasmid, wherein the microbes are auxotrophic, and wherein the ALAS variant encodes for the missing compound;
isolating surviving colonies;
inducing expression of the ALAS variant;
measuring the intensity of porphyrin fluorescence; and
comparing the intensity of porphyrin fluorescence to a baseline, wherein the comparison indicates whether the ALAS variant is hyperactive.

15. The method of claim 14, further wherein the baseline is wild type ALAS.

16. A method of increasing the enzymatic activity of 5-aminolevulinate synthase, comprising the steps of
providing a 5-aminolevulinate synthase precursor, wherein the precursor is DNA, cDNA, or mRNA; and
generating at least one mutation at the polypeptide or nucleic acid sequence that corresponds to the non-conserved residues within amino acids 422 to 439.

17. The ALAS enzyme of claim 1, wherein the active site loop amino acids are selected from the group consisting of SEQ ID No. 9, 10, 11, 12, 13, 14, 15, 16, and 17.

18. The method of claim 16, further comprising overexpressing the 5-aminolevulinate synthase variant in a target cell.

19. The method of claim 18, wherein the 5-aminolevulinate synthase is overexpressed by linking the 5-aminolevulinate synthase-encoding DNA under the control of a bacterial alkaline phosphatase promoter.

20. The method of claim 2, further comprising isolating the 5-aminolevulinate synthase variants-genetic complementation.

21. The method of claim 2, further comprising subcloning the 5-aminolevulinate synthase into a vector.

22. The method of claim 2, wherein the at least one mutation is generated by shuffling mutagenesis.
